(19) United States
(12) Patent Application Publication
(10) Pub. No.: US 2007/0122849 A1
(43) Pub. Date: May 31, 2007

(54) Rhesus monkey P2X7 purinergic receptor and uses thereof

(76) Inventors: Norbert T. Peekhaus, Harleysville, PA (US); Herbert Dang, Doylestown, PA (US); Le Thi Duong, Lansdale, PA (US)

Correspondence Address: MERCK AND CO., INC, P O BOX 2000, RAHWAY, NJ, 07065-0907, US

(21) Appl. No.: 11/605,677

(22) Filed: Nov. 29, 2006

Related U.S. Application Data
(60) Provisional application No. 60/740,447, filed on Nov. 29, 2005.

(51) Int. Cl. G01N 33/53; C07H 21/04; C12P 21/06; C07K 14/705; C12N 5/06; C12N 5/08

(52) U.S. Cl. 435/7.1; 435/69.1; 435/320.1; 435/325; 435/362; 435/369; 530/350; 536/23.5

(57) ABSTRACT

A P2X7 purinergic receptor has been isolated from the genome of Macaca mulatta (Rhesus monkey). Described is the amino acid sequence of the receptor, the nucleic acid encoding the receptor, and methods for using the receptor to identify analytes that are agonists or antagonists of the receptor.

### BACKGROUND OF THE INVENTION

(1) Field of the Invention

The present invention relates to the Macaca Mulatta (Rhesus monkey) P2X7 purinergic receptor, the nucleic acid encoding the receptor, and methods for identifying analytes that are agonists or antagonists of the receptor.

(2) Description of Related Art

P2X purinergic receptors are ligand gated ion channels, where upon the binding of nucleotides opens the channel resulting in the depolarization of the membrane via the influx of Ca2+. Of the seven P2X purinoceptor subtypes, the P2X7 is unique because the receptor functions not only as a cationic channel following brief activation, but prolonged or repeated activation yields to the formation of a large non-selective pore in plasma membrane. The P2X7 receptor is known to be highly expressed in neuronal cells, monocytes, macrophages and other hematopoietic cells. The most notable biological function is the processing of IL-1β cytokine, and as such is implicated as playing a key role in the degradation of cartilage, which is the hallmark of osteoarthritis. Mice deficient in P2X7 has been shown to have decreased nociception to neuropathic pain, suggesting that the P2X7 receptor may play a key role in pain processing. P2X7 may also have a role in autoimmune diseases such as rheumatoid arthritis.

The human P2X7 receptor has been described in U.S. Pat. Nos. 6,509,163 and 6,133,434 and in Surprenant et al., The cytolytic P_{2z} receptor for extracellular ATP identified as a P₂ₓ receptor P2X₇, Science 272: 735-738 (1996) and Hansen et al., Structural motif and characteristics of the extracellular domain of P₂ₓ receptors, Biochem. Biophys. Res. Comm. 236: 670-675 (1997). The following also relate to the P2X7 receptor and are of interest: Chessell et al., Disruption of the P2X7 purinoceptor gene abolishes chronic inflammatory and neuropathic pain, Pain: 114(3): 386-96 (1005); Baraldi et al., Agonists and antagonists acting at P2X7 receptor, Curr. Top. Med. Chem. 4(16): 1707-17 (2004); Wilson et al., Secretion of intracellular IL-1 receptor antagonist (type 1) is dependent on P2X7 receptor activation, J. Immunol. 173(2):1202-8 (2004); Kahlenberg and Dubyak., Mechanisms of caspase-1 activation by P2X7 receptor-mediated K⁺ release, Am. J. Physiol. Cell Physiol. 286(5): C1100-8 (2004); Alcaraz et al., Novel P2X7 receptor antagonists, Bioorg. Med. Chem. Lett. 13(22): 4043-6 (2003); Guerra et al., Purinergic receptor regulation of LPS-induced signaling and pathophysiology, J. Endotoxin. Res. 9(4): 256-63 (2003); and, North and Surprenant., Pharmacology of cloned P2X receptors, Annu. Rev. Pharmacol. Toxicol. 40: 563-80 (2000).

Therefore, in light of the above, antagonism of the P2X7 receptor may provide a new treatment for osteoarthritis by addressing the pain, as well as offering a method for modifying the disease process.

### BRIEF SUMMARY OF THE INVENTION

The present invention provides a Macaca Mulatta (Rhesus monkey) P2X7 purinergic receptor (rhP2X7), the nucleic acid encoding the rhP2X7 receptor, and methods for identifying analytes that are agonists or antagonists of the receptor.

Therefore, the present invention provides a nucleic acid comprising a nucleotide sequence encoding an rhP2X7 receptor. In further embodiments, the nucleic acid encodes an rhP2X7 has the amino acid sequence of SEQ ID NO:2. In further still embodiments, the nucleotide sequence of the nucleic acid comprises the nucleotide sequence of SEQ ID NO:1. in a further still embodiment, one or more of the nucleotide codons encoding the rhP2X7 receptor that occur at low frequency in nucleic acids encoding highly expressed proteins in humans have been replaced with nucleotide codons that occur at a higher frequency in the nucleic acids encoding the highly expressed proteins in humans.

The present invention further provides an expression vector comprising the nucleic acid encoding the rhP2X7 receptor operably linked to a promoter and further provides a host cell containing the expression vector therein.

The present invention further provides a process for producing the rhP2X7 receptor, which comprises culturing the host cell having the vector encoding the rhP2X7 in a cell culture medium under conditions for producing the rhP2X7 receptor. Thus, the present invention further provides the rhP2X7 receptor, in particular, the rhP2X7 receptor of claim 9 comprising the amino acid sequence of SEQ ID NO:2.

The present invention provides a method of screening an analyte for its ability to modulate the activity of a mammalian P2X7 receptor comprising contacting a recombinant, which expresses anrhP2X7 receptor, with a P2X7 receptor agonist, in the presence and absence of the analyte, and assaying for an alteration in the activity of the rhP2X7 receptor in the presence of the analyte, wherein a reduction or increase in the activity of the rhP2X7 receptor being indicative of an analyte that modulates mammalian P2X7 receptor activity. In particular embodiments, the agonist is ATP or BzATP.

The present invention further provides a method of screening a compound for its ability to enhance the activity of a mammalian P2X7 receptor comprising contacting a recombinant cell, which expresses an rhP2X7 receptor, with the analyte; assaying for activity of the rhP2X7 receptor; and, comparing the activity with the activity of the rhP2X7 receptor present in the absence of the analyte, wherein an increase in the activity of the rhP2X7 receptor in the presence of the analyte is indicative of an analyte that enhances mammalian P2X7 receptor activity.

The present invention further provides a method of screening an analyte for its ability to inhibit the activity of a mammalian P2X7 receptor comprising: contacting a recombinant cell, which expresses an rhP2X7 receptor, with the analyte and then with a P2X7 receptor agonist; assaying for activity of the rhP2X7 receptor; and, comparing the activity with the activity of the rhP2X7 receptor present in the absence of the analyte and in the presence of the agonist, wherein a decrease in the activity of the P2X7 receptor in the presence of the analyte is indicative of an analyte that inhibits P2X7 receptor activity. In particular embodiments, the agonist is ATP or Bz-ATP.

In further embodiments of the above methods, the assaying is effected by monitoring the uptake into the recombinant cell of a detectable molecule, for example, a fluorescent dye such as propidium iodide. In other embodiments, the assaying is effected by measuring the intracellular concentration of Ca²⁺ in the recombinant cell.

In further embodiments of the above methods, the recombinant cell is a HEK293 cell. In further embodiments, the rhP2X7 receptor has the amino acid sequence of SEQ ID NO:2.

As used throughout the specification and in the appended claims, the singular forms “a,” “an,” and “the” include the plural reference unless the context clearly dictates otherwise.

As used throughout the specification and appended claims, the following definitions and abbreviations apply.

The term “rhP2X7” means that the P2X7 receptor is of Rhesus monkey origin, either isolated from Rhesus monkey tissue, produced from a nucleic acid obtained from the monkey by recombinant means, produced from a nucleic acid synthesized in vitro but which encodes the P2X7 receptor, or synthesized in vitro. The term further includes biologically active fragments or portions of the PX7 receptor, including fusion or chimeric proteins.

The term “P2X7” means that the P2X7 receptor is of mammalian origin. The P2X7 receptor can be from another organism, for example, a mammal such as rat and mouse, or a human. The P2X7 receptor can either be isolated from tissue of the organism, produced from a nucleic acid obtained from the organism by recombinant means, produced from a nucleic acid synthesized in vitro but which encodes the P2X7 receptor, or synthesized in vitro. The term further includes biologically active fragments or portions of the P2X7 receptor, including fusion or chimeric proteins.

The term “promoter” refers to a transcription initiation region comprising a recognition site on a DNA strand to which the RNA polymerase binds. The promoter forms an initiation complex with RNA polymerase to initiate and drive transcriptional activity. The complex can be modified by activating sequences termed “enhancers” or inhibiting sequences termed “silencers”.

The term “gene” refers both to the genomic nucleic acid encoding the gene product, which for many genes comprises a combination of exon and intron sequences, and the cDNA derived from the mRNA encoding the gene product, which does not include intron sequences.

The term “cassette” or “expression cassette” refers to a nucleotide or gene sequence that is to be expressed from a vector, for example, the nucleotide or gene sequence encoding the rhP2X7 receptor. In general, a cassette comprises a gene sequence inserted into a vector which in some embodiments provides regulatory sequences for expressing the nucleotide or gene sequence. In other embodiments, the nucleotide or gene sequence provides the regulatory sequences for its expression. In further embodiments, the vector provides some regulatory sequences and the nucleotide or gene sequence provides other regulatory sequences. For example, the vector can provide a promoter for transcribing the nucleotide or gene sequence and the nucleotide or gene sequence provides a transcription termination sequence. The regulatory sequences which can be provided by the vector include, but are not limited to, enhancers, transcription termination sequences, splice acceptor and donor sequences, introns, ribosome binding sequences, and poly(A) addition sequences.

The term “vector” refers to some means by which DNA fragments can be introduced into a host organism or host tissue. There are various types of vectors including plasmids, viruses (including adenovirus), bacteriophages and cosmids.

The terms “polynucleotide”, “nucleic acid”, and “nucleic acid molecule” are intended to refer to any polymer of nucleotides bonded to one another by phosphodiester bonds, for example, ribonucleic acid (RNA) or deoxyribonucleic acid (DNA) molecules of any length. Polynucleotides or nucleic acid can include genes and fragments or portions thereof, probes, oligonucleotides, and primers. DNA can be either complementary DNA (cDNA) or genomic DNA, e.g. a gene encoding an rhP2X7 receptor or variant thereof. The terms “nucleic acid” and “polynucleotide” are used interchangeably herein.

The term “recombinant polynucleotide” or “recombinant nucleic acid” refers to a polynucleotide which has been modified by genetic engineering. The term also includes the fusion polypeptide comprising all or part of the rhP2X7 receptor fused to a heterologous polypeptide.

The term “variant thereof” refers to recombinant rhP2X7 receptor or polynucleotide. For example, a polynucleotide encoding an rhP2X7 receptor in which the codons have been optimized for enhanced expression in humans or a truncated form of the rhP2x7 receptor.

The phrases “codon-optimized”, “nucleotide codons are optimized for enhanced expression in humans”, “nucleotide sequence has been optimized for high expression”, and the like for describing the polynucleotides of the present invention mean that one or more of the nucleotide codons of the rhP2X7 receptor that occur at low frequency in nucleic acids encoding highly expressed proteins in an organism have been replaced with nucleotide codons that occur at a higher frequency in the nucleic acids encoding the highly expressed proteins in the organism. The nucleotide codon for a particular amino acid with “low frequency” is that nucleotide codon with the lowest frequency of use in nucleic acids that encode highly expressed proteins in the organism. The nucleotide codon for a particular amino acid with “high frequency” is that nucleotide codon with the highest frequency of use or a frequency of use that is higher than the nucleotide codon with the lowest frequency in nucleic acids that encode highly expressed proteins in the organism.

A “conservative amino acid substitution” refers to the replacement of one amino acid residue by another, chemically similar, amino acid residue. Examples of such conservative substitutions are: substitution of one hydrophobic residue (isoleucine, leucine, valine, or methionine) for another; substitution of one polar residue for another polar residue of the same charge (e.g., arginine for lysine; glutamic acid for aspartic acid).

The term “mammalian” refers to any mammal, including a human being.

The abbreviation “ORF” refers to the open reading frame of a gene.

The term “treatment” refers to both therapeutic treatment and prophylactic or preventative measures. Those in need of treatment include those already with the disorder as well as those prone to have the disorder or those in which the disorder is to be prevented.

A “disorder” is any condition, disease, or the like that would benefit from treatment with analytes identified by the methods described herein. This includes chronic and acute disorders or diseases including those pathological conditions which predispose the mammal to the disorder in question. Examples of disorders that may benefit from treatment with analytes identified by the methods disclosed herein include osteoarthritis and peripheral or neuropathic pain.

The term “analyte” includes molecule, compound, composition, drug, protein, peptide, nucleic acid, antibody and active fragment thereof, nucleic acid aptamer, peptide aptamer, and the like.

### BRIEF DESCRIPTION OF THE DRAWINGS

FIG. 1 show the nucleotide sequence encoding the rhP2X7 (SEQ ID NO:1).

FIG. 2 shows the amino acid sequence of the rhP2X7 (SEQ ID NO:2).

FIG. 3 shows a map of rhP2X7 (pcDNA5/FRT/TO/rhP2X7). The nucleotide sequence of pcDNA5/FRT/TO/rhP2X7 is shown in SEQ ID NO:7.

FIG. 4 shows the results of the change in membrane potential subsequent to activation with potent agonists (ATP, Bz-ATP) in HEK293 cells stably transfected with pcDNA5/FRT/TO/rhP2X7.

FIG. 5 shows the dose response curve for Bz-ATP in a calcium flux assay using the HEK293 cells stably transfected with pcDNA5/FRT/TO/rhP2X7 (384-well format).

FIG. 6 shows that the recombinant HEK293 cells stably transfected with pcDNA5/FRT/TO/rhP2X7 produced mRNA encoding the rhP2X7 receptor. Expression was measured relative to GAPDH expression.

FIG. 7 shows that various clones of recombinant HEK293 cells stably transfected with pcDNA5/FRT/TO/rhP2X7 produced rhP2X7. Lane (A), HEK293 wt; lane (B), HEK293-rhP2X7 clone 300; lane (C), HEK293-rhP2X7 clone 300 incubated in medium containing 10 μM tetracycline; lane (D), HEK293-rhP2X7 clone 212; lane (E), HEK293-rhP2X7 clone 212 incubated in medium containing 10 μM tetracycline; lane (F), HEK293-rhP2X7 clone 424; lane (G), HEK293-rhP2X7 clone 430; lane (H) HEK293-rhP2X7 clone 630; and, lane (I), HEK293-rhP2X7 clone 635.

### DETAILED DESCRIPTION OF THE INVENTION

The present invention provides nucleic acid molecules that encode the Macaca mulatta (Rhesus monkey) P2X7 (rhP2X7) purinergic receptor and provides methods for using the nucleic acid molecules and the rhP2X7 receptor produced therefrom in assays for identifying analytes (molecules, compounds, drugs, or compositions) that modulate the activity of the rhP2X7 receptor by interacting with or binding the rhP2X7 receptor or modulating the molecular or functional interaction between the rhP2X7 receptor and its ligand ATP. Modulators of the rhP2X7 receptor activity can be agonists, inverse agonists, or antagonists. Analytes that modulate activity of the P2X7 receptor can be useful for the treatment or prevention of osteoarthritis, various cardiovascular diseases, various immune diseases such as rheumatoid arthritis, various inflammatory diseases, as a treatment for peripheral or neuropathic pain and other disorders such as chronic obstructive pulmonary disease (COPD).

Non-limiting examples of methods for identifying such analytes include (i) cell-based binding methods for identifying analytes that bind the P2X7 receptor and inhibit or suppress binding between the P2X7 receptor and its ligand ATP, or interfere with transport of calcium or cell death induced by binding of the P2X7 receptor to its ligand ATP and (ii) cell-free binding methods for identifying analytes which bind the P2X7 receptor and inhibit or suppress binding between the P2X7 receptor and its ligand ATP. Thus, the present invention provides a means for identifying agonists and antagonists of the P2X7 receptor. The methods described herein are useful tools for identifying analytes which modulate molecular and/or functional interactions between the P2X7 receptor and its ligand ATP or and, therefore, are modulators of calcium transport or cell death induced by the interaction between the P2X7 receptor and its ligand ATP.

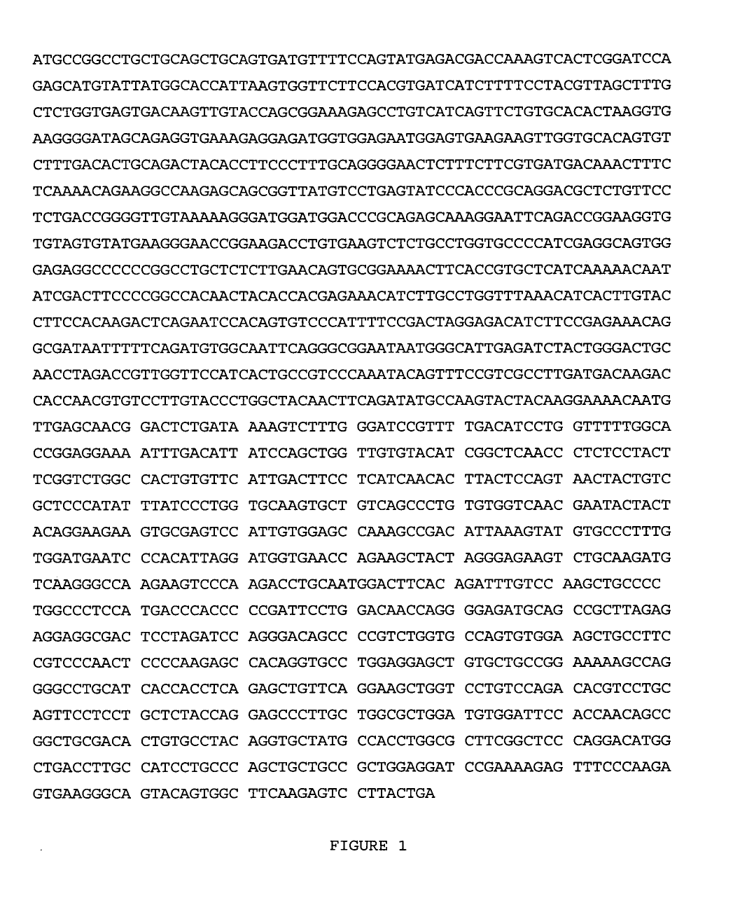

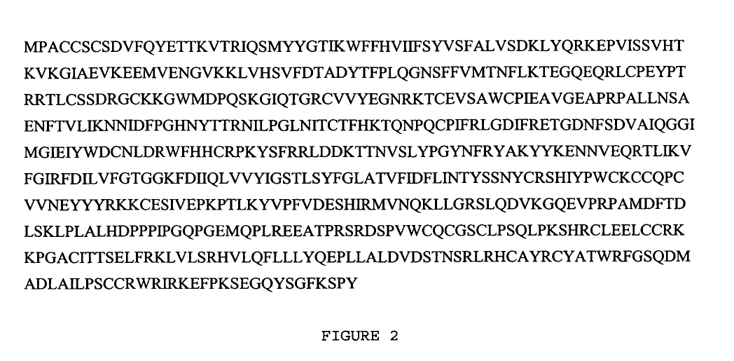

The present invention is particularly useful for identifying analytes of pharmaceutical importance which can be used to design or develop therapies or treatments for diseases or disorders which involve modulation of P2X7 receptor activity. Therefore, in one aspect of the present invention, an isolated nucleic acid molecule is provided which comprises a sequence of nucleotides encoding an RNA molecule that can be translated in vivo or in vitro to produce an rhP2X7 receptor with the amino acid sequence as set forth in SEQ ID NO:2 (FIG. 2). In further embodiments, the nucleic acid is substantially free from other nucleic acids of the Rhesus monkey or substantially free from other nucleic acids. In a further embodiment, the isolated nucleic acid molecule encoding the rhP2X7 receptor comprises the nucleotide sequence set forth in SEQ ID NO:1 (FIG. 1).

The isolated nucleic acid molecules include both deoxyribonucleic acid (DNA) and ribonucleic acid (RNA) molecules encoding the rhP2X7 receptor. The isolated nucleic acid molecules further include genomic DNA and complementary DNA (cDNA) encoding the rhP2X7 receptor, either of which can be single- or double-stranded, as well as synthetic DNA, such as a synthesized, single stranded polynucleotide. When single-stranded, the DNA molecule can comprise either the coding (sense) strand or the non-coding (antisense) strand. For most cloning purposes, DNA is a preferred nucleic acid.

In further aspects of the present invention, modified rhP2X7 receptors are provided which have an amino acid sequence, which is substantially similar to the amino acid sequence set forth in SEQ ID NO:2, and nucleic acids, which encode the rhP2X7 receptor, for use in the analyte screening assays disclosed herein. Further provided are nucleic acids encoding the rhP2X7 receptor which have a nucleotide sequence substantially similar to the nucleotide sequence set forth in SEQ ID NO:1. As used herein, the term “substantially similar” with respect to SEQ ID NO:2 means that the rhP2X7 receptor contains mutations such as amino acid substitution or deletion mutations that do not abrogate the ability of the rhP2X7 receptor to bind its ligand. The mutations include naturally occurring allelic variants and variants produced by recombinant DNA methods, for example fusion proteins comprising a portion of the rhP2X7 fused to another protein or polypeptide. As used herein, the term “substantially similar” with respect to SEQ ID NO:1 means that the rhP2X7 receptor encoded by the nucleic acid contains mutations such as nucleotide substitution or deletion mutations which do not abrogate the ability of the rhP2X7 receptor to bind its ligand. The mutations include naturally occurring allelic variants and variants produced by recombinant DNA methods, for example optimizing the codons encoding the rhP2X7 receptor for enhanced expression in cells of human origin. In general, any of the foregoing mutations which do not abrogate the ability of the rhP2X7 receptor to bind its ligand are conservative mutations.

The present invention further includes biologically active mutants of SEQ ID NO:1. In general, any such biologically active mutant will encode either a polypeptide, which has properties or activity substantially similar to the properties or activity of the rhP2X7 receptor, including but not limited to the rhP2X7 receptor as set forth in SEQ ID NO:2. Any such polynucleotide includes, but is not limited to, nucleotide substitutions, deletions, additions, amino-terminal truncations, and carboxy-terminal truncations which do not substantially abrogate the properties or activities of the rhP2X7 receptor produced therefrom. Thus, the mutations of the present invention encode mRNA molecules that express an rhP2X7 receptor in a eukaryotic cell which has sufficient activity (ability to bind one or more of its receptors) to be useful in drug discovery. Further, the present invention provides biologically active fragments of SEQ ID NO:2 and mutants thereof and the DNA encoding such fragments. The biologically active fragments can include any combination of the ligand binding domain, transmembrane domain, and G protein binding domain. For example, the biologically active fragment can consist of the ligand binding domain and the transmembrane domain.

The present invention further includes synthetic DNAs (sDNA) which encode the rhP2X7 receptor wherein the nucleotide sequence of the sDNA differs from the nucleotide sequence of SEQ ID NO:1 but still encodes rhP2X7 receptor as set forth in SEQ ID NO:2 or mutant with substantially similar properties or activity. For example, to express or enhance expression of the rhP2X7 receptor in a particular cell type (for example, see Lathe, Synthetic Oligonucleotide Probes Deduced from Amino Acid Sequence Data: Theoretical and Practical Considerations, J. Molec. Biol. 183: 1-12 (1985); Nakamura et al., Nuc. Acid Res. 28: 292 (2000); Fuglsang, Protein Expression & Purification 31: 247-249 (2003)), it may be necessary to change the sequence comprising one or more of the codons encoding the rhP2X7 receptor to sequences to enable expression of the rhP2X7 receptor in the particular cell type. Such changes include modifications for codon usage peculiar to a particular host or removing cryptic cleavage or regulatory sites which would interfere with expression of the rhP2X7 receptor in a particular cell type. Therefore, the present invention discloses codon redundancies which may result in numerous DNA molecules expressing an identical protein. For purposes of this specification, a sequence bearing one or more replaced codons will be defined as a degenerate variation. Also included within the scope of this invention are mutations either in the DNA sequence or the translated protein that do not alter or do not substantially alter the ultimate physical or functional properties of the expressed protein (in general, these mutations are referred to as conservative mutations). For example, substitution of valine for leucine, arginine for lysine, or asparagine for glutamine may not cause a change in the functionality of the polypeptide.

Included in the present invention are DNA sequences that hybridize to SEQ ID NO:1 under stringent conditions. By way of example, and not limitation, a procedure using conditions of high stringency is as follows. Prehybridization of filters containing DNA is carried out for about two hours to overnight at about 65° C. in buffer composed of 6×SSC, 5× Denhardt's solution, and 100 μg/mL denatured salmon sperm DNA. Filters are hybridized for about 12 to 48 hrs at 65° C. in prehybridization mixture containing 100 μg/mL denatured salmon sperm DNA and labeled DNA (for example, 5 to 20×10⁶ cpm of ³²P-labeled DNA). The filters are washed at 37° C. for about 1 hour in a solution containing 2×SSC, 0.1% SDS. This is followed by a wash in 0.1×SSC, 0.1% SDS at 50° C. for 45 minutes before autoradiography. Other procedures using conditions of high stringency would include either a hybridization step carried out in 5×SSC, 5× Denhardt's solution, 50% formamide at about 42° C. for about 12 to 48 hours or a washing step carried out in 0.2×SSPE, 0.2% SDS at about 65° C. for about 30 to 60 minutes. Reagents mentioned in the foregoing procedures for carrying out high stringency hybridization are well known in the art. Details of the composition of these reagents can be found in Sambrook et al., Molecular Cloning: A Laboratory Manual 2^{nd} Edition; Cold Spring Harbor Laboratory Press, Cold Spring Harbor, N.Y., (1989) or Sambrook and Russell, Molecular Cloning: A Laboratory Manual, 3rd Edition. Cold Spring Harbor Laboratory Press, Plainview, N.Y. (2001). In addition to the foregoing, other conditions of high stringency which may be used are also well known in the art.

In an another aspect of the present invention, a substantially purified form of an rhP2X7 receptor which comprises a sequence of amino acids as disclosed in FIG. 2 (SEQ ID NO:2) is provided. Further provided are biologically active fragments and/or mutants of the rhP2X7 receptor, which comprise at least a portion of the amino acid sequence set forth in SEQ ID NO: 2. These mutations or fragments include, but not limited to, amino acid substitutions, deletions, additions, amino terminal truncations, and carboxy-terminal truncations such that these mutations provide for proteins or protein fragments of diagnostic, therapeutic, or prophylactic use and are useful for screening assays for identifying analytes that interfere with the interaction of the rhP2X7 receptor and its ligand, such analytes being useful for treatment of diseases or disorders which involve modulation of rhP2X7 receptor activity. In a particular embodiment, the present invention provides an isolated nucleic acid molecule comprising a sequence that encodes a mutated rhP2X7 receptor comprising the sequence set forth in SEQ ID NO:2 with about 1 to 10 amino acid additions, deletions, or substitutions, wherein the mutated rhP2X7 receptor polypeptide is capable of binding its ligand.

The rhP2X7 receptors of the present invention can be the “mature” protein or a fragment or portion thereof (e.g., extracellular domain, intracellular 1 or 2 domains, or transmembrane 1 or 2 domains), any of which can be a part of a larger protein such as a fusion protein. It is often advantageous to include covalently linked to the amino acid sequence of the rhP2X7 receptor, an additional amino acid sequence which contains secretory or leader sequences, pro-sequences, sequences which aid in purification of the rhP2X7 receptors such as multiple histidine residues (polyHis) or antibody-binding epitopes, or one or more additional amino acid sequences which confer stability to the rhP2X7 receptor during recombinant production. Thus, rhP2X7 receptor fusion proteins are provided which comprise all or part of the rhP2X7 receptor linked at its amino or carboxyl terminus to proteins or polypeptides such as green fluorescent protein (GFP), c-myc epitope, alkaline phosphatase, protein A or G, glutathione S-transferase (GST), polyHis, peptide cleavage site, or antibody Fc region. Any such fusion construct can be expressed in a cell line of interest and used to screen for modulators of the rhP2X7 receptor disclosed herein. In a particular embodiment, the present invention provides an isolated nucleic acid molecule comprising a sequence that encodes a fusion rhP2X7 receptor comprising the sequence set forth in SEQ ID NO:2 or a fusion protein with amino acid additions, deletions, or substitutions, wherein the mutated rhP2X7 receptor is capable of binding its ligand.

The present invention further provides vectors which comprise at least one of the nucleic acid molecules disclosed throughout this specification, preferably wherein the nucleic acid molecule is operably linked to a heterologous promoter. These vectors can comprise DNA or RNA. For most cloning purposes, DNA plasmid or viral expression vectors are preferred. Typical expression vectors include plasmids, modified viruses, bacteriophage, cosmids, yeast artificial chromosomes, and other forms of episomal or integrated DNA, any of which expresses the rhP2X7 receptor, polypeptide fragment thereof, or fusion protein comprising all or part of the rhP2X7 receptor encoded therein. It is well within the purview of the skilled artisan to determine an appropriate vector for a particular gene transfer or other use. As used herein, the term “recombinant rhP2X7 receptor” is intended to include any variation of rhP2X7 receptor disclosed herein that is expressed from a vector transfected into a eukaryote cell or transformed into a prokaryote cell. Transfected eukaryote cells and transformed prokaryote cells are referred to as recombinant host cells.

An expression vector containing DNA encoding an rhP2X7 receptor or any one of the aforementioned variations thereof wherein the DNA is preferably operably linked to a heterologous promoter can be used for expression of the recombinant rhP2X7 receptor in a recombinant host cell. Such recombinant host cells can be cultured under suitable conditions to produce recombinant rhP2X7 receptor or a biologically equivalent form, for example, as shown in the Examples. Expression vectors include, but are not limited to, cloning vectors, modified cloning vectors, specifically designed plasmids, or specifically designed viruses.

Commercially available mammalian expression vectors which are suitable for recombinant rhP2X7 receptor expression include, but are not limited to, pcDNA3.neo (Invitrogen, Carlsbad, Calif.), pcDNA3.1 (Invitrogen, Carlsbad, Calif.), pcDNA3.1/Myc-His (Invitrogen), pCI-neo (Promega, Madison, Wis.), pLITMUS28, pLITMUS29, pLITMUS38 and pLITMUS39 (New England Biolabs, Beverly, Mass.), pcDNAI, pcDNAIamp (Invitrogen), pcDNA3 (Invitrogen), pMC1neo (Stratagene, La Jolla, Calif.), pXT1 (Stratagene), pSG5 (Stratagene), EBO-pSV2-neo (ATCC 37593) pBPV-1(8-2) (ATCC 37110), pdBPV-MMTneo (342-12) (ATCC 37224), pRSVgpt (ATCC 37199), pRSVneo (ATCC 37198), pSV2-dhfr (ATCC 37146), pUCTag (ATCC 37460), and IZD35 (ATCC 37565).

Also, a variety of bacterial expression vectors can be used to express recombinant rhP2X7 receptor in bacterial cells. Commercially available bacterial expression vectors which may be suitable for recombinant rhP2X7 receptor expression include, but are not limited to, pCR2.1 (Invitrogen), pET11a (Novagen, Madison, Wis.), lambda gt11 (Invitrogen), and pKK223-3 (Pharmacia).

In addition, a variety of fungal cell expression vectors may be used to express recombinant rhP2X7 receptor in fungal cells. Commercially available fungal cell expression vectors which are suitable for recombinant rhP2X7 receptor expression include, but are not limited to, pYES2 (Invitrogen) and Pichia expression vector (Invitrogen).

Also, a variety of insect cell expression vectors can be used to express recombinant rhP2X7 receptor in insect cells. Commercially available insect cell expression vectors which can be suitable for recombinant expression of rhP2X7 receptor include, but are not limited to, pBlueBacIII and pBlueBacHis2 (Invitrogen), and pAcG2T (Pharmingen).

Viral vectors which can be used for expression of recombinant rhP2X7 receptor include, but are not limited to, adenovirus vectors, adeno-associated virus vectors, herpesvirus vectors, Sindbis virus vectors, Simliki forest virus vectors, pox virus vectors (such as vaccinia virus, fowl pox, canary pox, and the like), retrovirus vectors, and baculovirus vectors. Many of viral vectors are commercially available.

The nucleic acids of the present invention in the above vectors for expressing the rhP2X7 receptor or fragment thereof are preferably assembled into an expression cassette that comprises sequences which provide for efficient expression of the rhP2X7 receptor or variant thereof encoded thereon in a eukaryote cell, preferably a mammalian cell such as a CHO cell or variant thereof. The cassette preferably contains the full-length cDNA encoding the rhP2X7 receptor or a DNA encoding a fragment of the rhP2X7 receptor with homologous or heterologous transcriptional and translational control sequences operably linked to the DNA. Such control sequences include at least a transcription promoter (constitutive or inducible) and transcription termination sequences and can further include other regulatory elements such as transcription enhancers, ribosome binding sequences, splice junction sequences, and the like. In most embodiments, the promoter is a heterologous promoter; however, in particular embodiments, the promoter can be the natural rhP2X7 receptor promoter. In either embodiment, the expression cassette allows for ectopic expression of the rhP2X7 receptor in various host cells of non-African green monkey origin. In a particularly useful embodiment, the promoter is the constitutive cytomegalovirus immediate early promoter with or without the intron A sequence (CMVᵢₑ promoter) although those skilled in the art will recognize that any of a number of other known promoters such as the strong immunoglobulin promoter, Rous sarcoma virus long terminal repeat promoter, SV40 small or large T antigen promoter, or the like. A transcriptional terminator can be the bovine growth hormone terminator although other known transcriptional terminators such as SV40 termination sequences can also be used.

The present invention further provides recombinant host cells transformed or transfected with a vector comprising any one of the aforementioned nucleic acid molecules, particularly host cells transformed or transfected with a vector comprising any one of the aforementioned nucleic acid molecules wherein the nucleic acid molecule is operably linked to a promoter. Recombinant host cells include bacteria such as E. coli, fungal cells such as yeast, plant cells, mammalian cells including, but not limited to, cell lines of bovine, porcine, monkey, human, or rodent origin; and insect cells including, but not limited to, Drosophila and silkworm-derived cell lines. For instance, one insect expression system utilizes Spodoptera frugiperda (Sf21) insect cells (Invitrogen) in tandem with a baculovirus expression vector (pAcG2T, Pharmingen, San Diego, Calif.). Mammalian cells which may be suitable and which are commercially available, include but are not limited to, L cells L-M(TK-) (ATCC CCL-1.3), L cells L-M (ATCC CCL-1.2), Saos-2 cells (ATCC HTB-85), 293 cells (ATCC CRL-1573), Raji cells (ATCC CCL-86), CV-1 cells (ATCC CCL-70), COS-1 cells (ATCC CRL-1650), COS-7 cells (ATCC CRL-1651), CHO-K1 cells (ATCC CCL-61), 3T3 cells (ATCC CCL-92), NIH/3T3 cells (ATCC CRL-1658), HeLa cells (ATCC CCL-2), C127I cells (ATCC CRL-1616), BS-C-1 cells (ATCC CCL-26), MRC-5 cells (ATCC CCL-171), HEK293T cells (ATCC CRL-1573), ST2 cells (Riken Cell bank, Tokyo, Japan RCB0224), C3H10T1/2 cells (JCRB0602, JCRB9080, JCRB0003, or IFO50415), and CPAE cells (ATCC CCL-209). Such recombinant host cells can be cultured under suitable conditions to produce rhP2X7 receptor or a biologically equivalent form. Recombinant eukaryote cells include both transiently infected cells and stably transfected cells in which the expression cassette or vector is integrated into the genome of the cell.

As noted above, an expression vector containing DNA encoding rhP2X7 receptor or any one of the aforementioned variations thereof can be used to express the rhP2X7 receptor encoded therein in a recombinant host cell. Therefore, the present invention provides a process for expressing an rhP2X7 receptor or any one of the aforementioned variations thereof in a recombinant host cell comprising introducing the vector comprising a nucleic acid that encodes the rhP2X7 receptor into a suitable host cell and culturing the host cell under conditions which allow expression of the rhP2X7 receptor and preferably, integration of the rhP2X7 receptor into the cell's membrane. In a further embodiment, the rhP2X7 receptor has an amino acid sequence substantially as set forth in SEQ ID NO:2 and binds at least its ligand, and the nucleic acid encoding the rhP2X7 receptor is operably linked to a heterologous promoter which can be constitutive or inducible. Thus, the present invention further provides a cell comprising a nucleic acid encoding the rhP2X7 receptor which has an amino acid sequence substantially as set forth in SEQ ID NO:2, which preferably binds at least its ligand, and wherein the nucleic acid encoding the rhP2X7 receptor is operably linked to a heterologous promoter.

Following expression of rhP2X7 receptor or any one of the aforementioned variations of the rhP2X7 receptor in a host cell, rhP2X7 receptor or variant thereof can be recovered to provide rhP2X7 receptor in a form capable of binding to its ligand. Several rhP2X7 receptor purification procedures are available and suitable for use. The rhP2X7 receptor can be purified from cell lysates and extracts by various combinations of, or individual application of, salt fractionation, ion exchange chromatography, size exclusion chromatography, hydroxylapatite adsorption chromatography, or hydrophobic interaction chromatography. In addition, rhP2X7 receptor can be separated from other cellular polypeptides by use of an immunoaffinity column made with monoclonal or polyclonal antibodies specific for rhP2X7 receptor or a particular epitope thereof. Alternatively, in the case of fusion polypeptides comprising all or a portion of the rhP2X7 receptor fused to a second polypeptide, purification can be achieved by affinity chromatography comprising a reagent specific for the second polypeptide such as an antibody or metal.

Cloning, expression vectors, transfections and transformations, and protein isolation of expressed proteins are well known in the art and have been described, for example, in Sambrook et al., Molecular Cloning: A Laboratory Manual 2^{nd} Edition; Cold Spring Harbor Laboratory Press, Cold Spring Harbor, N.Y., (1989) or Sambrook and Russell, Molecular Cloning: A Laboratory Manual, 3rd Edition. Cold Spring Harbor Laboratory Press, Plainview, N.Y. (2001). For example, any of a variety of procedures may be used to clone DNA encoding rhP2X7 receptor from RNA isolated from the monkey. These methods include, but are not limited to, the method shown in the Examples.

The DNA molecules, RNA molecules, and recombinant polypeptides of the present invention can be used to screen and measure levels of rhP2X7 receptor expression in homologous or heterologous cells. The recombinant polypeptides, DNA molecules, and RNA molecules lend themselves to the formulation of kits suitable for the detection and typing of rhP2X7 receptors. Such a kit would comprise a compartmentalized carrier suitable to hold in close confinement at least one container. The carrier would further comprise reagents such as recombinant rhP2X7 receptor or anti-rhP2X7 receptor antibodies suitable for detecting rhP2X7 receptors. The carrier may also contain a means for detection such as labeled antigen or enzyme substrates or the like. The kit enables identification of polymorphic forms of rhP2X7 receptor which can then be used in the previously described methods to determine the effect the polymorphism has on binding between the polymorphic rhP2X7 receptor and its ligand.

In accordance with yet another embodiment of the present invention, there are provided antibodies having specific affinity for the rhP2X7 receptor or epitope thereof. The term “antibodies” is intended to be a generic term which includes polyclonal antibodies, monoclonal antibodies, Fab fragments, single V_{H} chain antibodies such as those derived from a library of camel or llama antibodies or camelized antibodies (Nuttall et al., Curr. Pharm. Biotechnol. 1: 253-263 (2000); Muyldermans, J. Biotechnol. 74: 277-302 (2001)), and recombinant antibodies. The term “recombinant antibodies” is intended to be a generic term which includes single polypeptide chains comprising the polypeptide sequence of a whole heavy chain antibody or only the amino terminal variable domain of the single heavy chain antibody (V_{H} chain polypeptides) and single polypeptide chains comprising the variable light chain domain (V_{L}) linked to the variable heavy chain domain (V_{H}) to provide a single recombinant polypeptide comprising the Fv region of the antibody molecule (scFv polypeptides)(See, Schmiedl et al., J. Immunol. Meth. 242: 101-114 (2000); Schultz et al., Cancer Res. 60: 6663-6669 (2000); Dubel et al., J. Immunol. Meth. 178: 201-209 (1995); and in U.S. Pat. No. 6,207,804 B1 to Huston et al.). Construction of recombinant single V_{H} chain or scFv polypeptides which are specific against an analyte can be obtained using currently available molecular techniques such as phage display (de Haard et al., J. Biol. Chem. 274: 18218-18230 (1999); Saviranta et al., Bioconjugate 9: 725-735 (1999); de Greeff et al., Infect. Immun. 68: 3949-3955 (2000)) or polypeptide synthesis. In further embodiments, the recombinant antibodies include modifications such as polypeptides having particular amino acid residues or ligands or labels such as horseradish peroxidase, alkaline phosphatase, fluors, and the like. Further still embodiments include fusion polypeptides which comprise the above polypeptides fused to a second polypeptide such as a polypeptide comprising protein A or G.

The antibodies specific for rhP2X7 receptor can be produced by methods known in the art. For example, polygonal and monoclonal antibodies can be produced by methods well known in the art, as described, for example, in Harlow and Lane, Antibodies: A Laboratory Manual. Cold Spring Harbor Laboratory Press: Cold Spring Harbor, N.Y. (1988). The rhP2X7 receptor or fragments thereof can be used as immunogens for generating such antibodies. Alternatively, synthetic peptides can be prepared (using commercially available synthesizers) and used as immunogens. Amino acid sequences can be analyzed by methods well known in the art to determine whether they encode hydrophobic or hydrophilic domains of the corresponding polypeptide. Altered antibodies such as chimeric, humanized, camelized, CDR-grafted, or bifunctional antibodies can also be produced by methods well known in the art. Such antibodies can also be produced by hybridoma, chemical synthesis or recombinant methods described, for example, in Sambrook et al., supra., and Harlow and Lane, supra. Both anti-peptide and anti-fusion protein antibodies can be used. (See, for example, Bahouth et al., Trends Pharmacol. Sci. 12: 338 (1991); Ausubel et al., Current Protocols in Molecular Biology (John Wiley and Sons, N.Y. (1989)).

Antibodies so produced can be used for the immunoaffinity or affinity chromatography purification of the rhP2X7 receptor or rhP2X7 receptor/ligand complexes. The above referenced anti-rhP2X7 receptor antibodies can also be used to modulate the activity of the rhP2X7 receptor in living animals, in humans, or in biological tissues isolated therefrom. Accordingly, contemplated herein are compositions comprising a carrier and an amount of an antibody having specificity for rhP2X7 receptor effective to block naturally occurring rhP2X7 receptor from binding its ligand.

Therefore, the nucleic acids encoding rhP2X7 receptor or variant thereof, vectors containing the same, host cells transformed with the nucleic acids or vectors which express the rhP2X7 receptor or variants thereof, the rhP2X7 receptor and variants thereof, as well as antibodies specific for the rhP2X7 receptor, can be used in in vivo or in vitro methods for screening a plurality of analytes to identify analytes that are modulators of the rhP2X7 activity and receptor/ligand interaction. These methods provide information regarding the function and activity of the rhP2X7 receptor and variants thereof which can lead to the identification and design of molecules, compounds, or compositions capable of specific interactions with Rhesus monkey and ultimately, the human P2X7 receptor. In preferred embodiments, the methods identify analytes which interfere with the binding of the rhP2X7 receptor to its ligand or activity of the rhP2X7 receptor. Such analytes are useful either alone or in combination with other compounds for treating a wide variety of psychiatric or neurological diseases or disorders. Accordingly, the present invention provides methods (screening assays) for identifying analytes that modulate the binding of rhP2X7 receptor to its ligand or activity of the rhP2X7 receptor and which can be used for treating the aforementioned diseases or disorders. The method involves identifying analytes that bind to the rhP2X7 receptor and/or have a stimulatory or inhibitory effect on the biological activity of the rhP2X7 receptor or its expression and then determining which of these analytes has an effect on symptoms or diseases regarding the aforementioned disorders and diseases in an in vivo assay.

The screening assays include (i) cell-based methods for identifying analytes which bind the rhP2X7 receptor, inhibit or suppress binding between an rhP2X7 receptor and its ligand, or modulate activity of the rhP2X7 receptor, and (ii) cell-free methods for identifying analytes which bind the rhP2X7 receptor, inhibit or suppress binding between the rhP2X7 receptor and its ligand, or modulate activity of the rhP2X7 receptor. Analytes that bind or modulate activity of the rhP2X7 receptor include both agonists and antagonists. U.S. Pat. No. 6,509,163 discloses methods of screening for modulators of P2X7 receptors.

In one embodiment of a cell-based method, a host cell transformed with an rhP2X7 receptor encoding sequence is contacted with an agonist, for example, ATP or Bz-ATP, in the presence and absence of a test analyte. The effect of the test analyte on the activation of the receptor is then determined, for example, using the membrane potential or calcium flux assays described in the Examples. Analytes that inhibit receptor activation are likely to be of value in disorders of the nervous system (particularly those diseases with a component of chronic inflammation, such as Alzheimer's disease), diseases involving acute or chronic inflammation (including but not limited to rheumatoid arthritis, amyloidosis, bacterial, viral and other microbial infections), osteoarthritis, and disorders of the hematopoietic system and immune response (including but not limited to autoimmune disorders, allergies and lymphoproliferative disorders), and diseases involving apoptotic cell death, such as cardiac and cerebral ischemia. Analytes that activate rhP2X7 receptors are likely to be of value in combating microbial infections, particularly tuberculosis.

Analytes selected using the above-described method can be formulated as pharmaceutical compositions using known methods. Appropriate administration regimens can be established by one skilled in the art.

The following examples are intended to promote a further understanding of the present invention.

### EXAMPLE 1

The cDNA encoding the rhP2X7 receptor was cloned using standard RT-PCR methods.

The template and PCR primers were as follows. The PCR primer P2X7uphind had the nucleotide sequence 5′-CGCAAGCTTA TGCCGGCCTG CTGCAGCTGC AGTG-3′ (SEQ ID NO:3), the PCR primer P2X7dohind had the nucleotide sequence 5′-CGCAAGCTTT CAGTAAGGAC TCTTGAAGCC ACT-3′ (SEQ ID NO:4), the PCR primer P2X7ecomidup had the nucleotide sequence 5′-CGCGAATTCC TTTGCTCTGC GGGTCCATCC ATC-3′ (SEQ ID NO:5) and the PCR primer P2X7ecomiddo had the nucleotide sequence 5′-CGCGAATTCA GACCGGAAGG TGTGTAGTGT ATGAAG-3′ (SEQ ID NO:6).

The cDNA fragment (1.8 kb) encoding the entire rhP2X7 receptor was PCR amplified from Rhesus monkey RNA as follows. The above two sets of PCR primers were used to generate two overlapping DNA fragments: a 1,350 bp EcoRI-HindIII DNA fragment and a 450 bp HindIII-EcoRI DNA fragment. The DNA fragments overlapped at the EcoRI site in the nucleotide sequence encoding the rhP2X7 receptor. Stratagene PCR kit (Stratagene, La Jolla, Calif.) was used and the rhesus cDNA library from OriGene (Rockville, Md.) was used. PCR amplification conditions were denaturation at 95° C. followed by 35 cycles at 95° C. for 15 seconds and 58° C. for 1.5 minute.

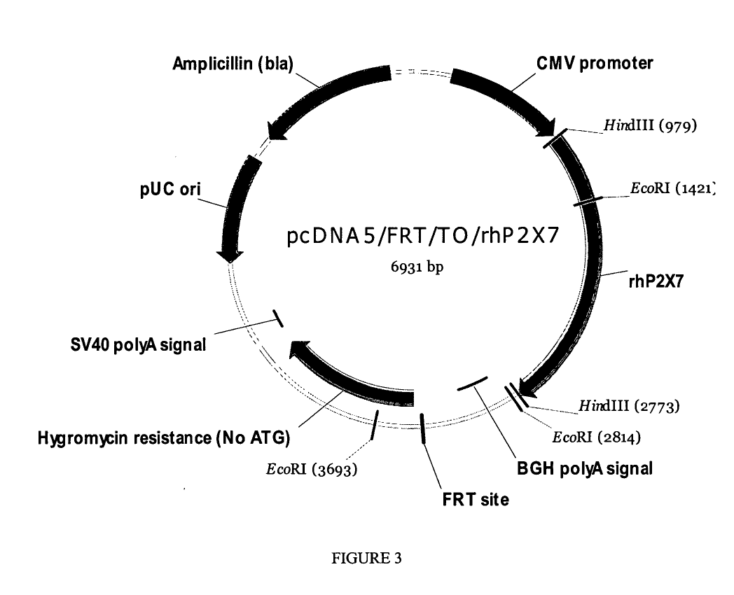

Both PCR amplified DNA fragments were separately cloned into pBR322 digested with EcoRI and HindIII. The rhesus P2X7 DNA fragments were excised from the pBR322 plasmid using the restriction enzymes EcoRI and HindIII and the DNA fragments subcloned into the HindIII site of the expression vector pcDNA5/FRT/TO (Invitrogen, La Jolla, Calif.) to produce plasmid pcDNA5/FRT/TO/rhP2X7 containing a full length cDNA encoding the rhP2X7. Both strands of the cDNA clone were then sequenced. The nucleotide sequence (SEQ ID NO:1) is shown in FIG. 1 and the amino acid sequence (SEQ ID NO:2) is shown in FIG. 2. A map of the vector pcDNA5/FRT/TO containing rhP2X7 in the proper orientation for expression of the rhP2X7 (pcDNA5/FRT/TO/rhP2X7) is shown in FIG. 3. The nucleotide sequence of pcDNA5/FRT/TO/rhP2X7 is set forth in SEQ ID NO:7.

### EXAMPLE 2

A recombinant HEK293 cell line containing the rhP2X7 receptor stably integrated into the genome of the cells was constructed as follows.

FLP-IN T-REX 293 (recombinant HEK293 host cells that stably express the lacZ-Zeocin and contain a single integrated FRT site (Invitrogen)) were co-transfected with pcDNA5/FRT/TO/rhP2X7 and the plasmid pOG44 (a Flp-recombinase expression vector, Invitrogen) using LIPOFECTAMINE 2000 (Invitrogen) and standard transfection protocols for producing stably transfected cells from the manufacturer (Invitrogen). The recombinant cells contained an SV40 promoter and ATG adjacent to the single FRT recombination site. Therefore, the FRT recombination site allows expression of the ATG minus hygromycin gene provided by pcDNA5/FRT/TO when recombined into the FRT recombination site. Stably transfected recombinant HEK293 cells expressing recombinant rhP2X7 were selected through growth in media containing hygromycin and blasticin.

### EXAMPLE 3

RhP2X7 receptor activity in the stably transfected HEK293 cells was monitored by measuring calcium flux and change in membrane potential subsequent to activation with potent agonists (ATP, Bz-ATP). The membrane potential was measured using the following protocol.

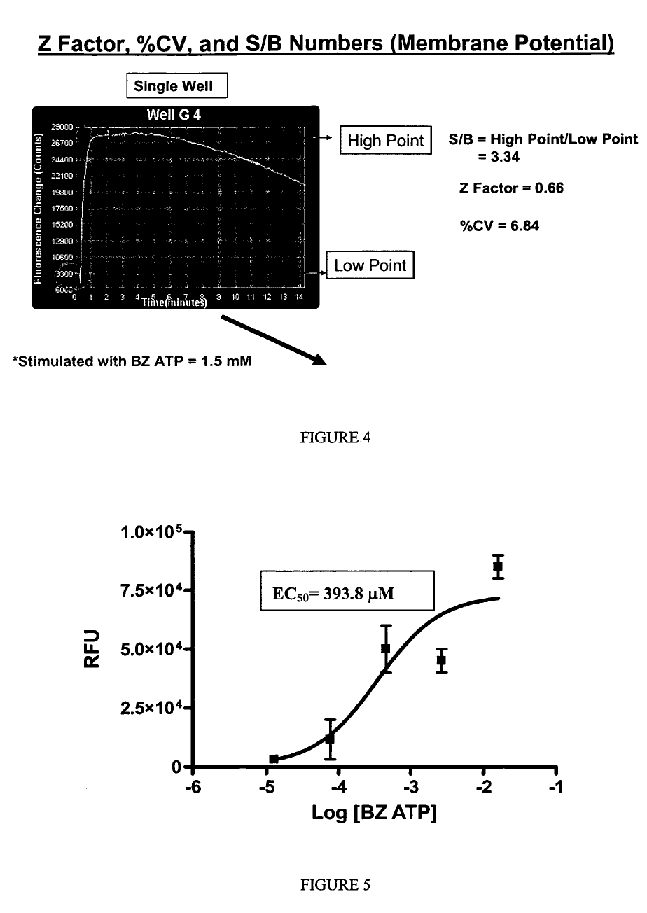

Stably transfected recombinant HEK293 cells were plated into the wells of a 384-well Costar plates (25 μL/well) in growth medium (DMEM without pyruvate (Invitrogen-11965-084) with 10% FBS (Invitrogen 10082-139), 100 U/ml P/S (Invitrogen 15140-148), and 100 ug/mL hygromycin (Roche, 843555) and the plates incubated for 24 hours at 37° C., 5% CO₂. Then, 25 μL of membrane potential buffer (Blue dye, Molecular Devices, Sunnyvale, Calif.) was added to the medium and the cells incubated for 30 minutes at 37° C., 5% CO₂. Next, about 30 to 100 nL of either analyte or antagonist control or 10 μL agonist 3′-O-(4-benzoylbenzoyl)-ATP (Bz-ATP) (at an EC₈₀) was added to the medium in separate wells of the plates. After incubating the plates for 30 minutes at 37° C., 5% CO₂, fluorescence was read using a fluorometric plate reader (FLIPR, Molecular Devices). Data were analyzed using the formula EC₅₀=using (Max−Min); Signal to Noise Ratio (S/B)=Max/Min; Z′ Factor=1−[3*SD of Max)+(3*SD of Min)/(mean of Max−mean of Min). The results are shown in FIG. 4.

Calcium flux was measured using the following FLIPR Calcium Flux Assay protocol (Molecular Devices, Sunnyvale, Calif.). Cells were plated into the wells of 384-well Costar plates (25 μL per well) and incubated for 24 hours at 37° C., 5% CO₂. Then, about 20 μL of FLUO-4 loading buffer containing 1% Propenecid (Fluorescent calcium indicator from Invitrogen) was added to each of the wells of the plates and the plates incubated for 60 minutes at 37° C., 5% CO₂. Next, about 30 to 100 nL of either analyte or antagonist control or 10 μL agonist Bz-ATP (at an EC₈₀) was added to the medium in separate wells of the plates. After incubation for 30 minutes at 37° C., fluorescence was read using a fluorometric plate reader (FLIPR, Molecular Devices). FLUO-4 was excited at 488 nm, and fluorescence was measured at 510 nm in a time-resolved mode (1-Hz frequency). Relative f/f0 intensity (in counts/ms) was used as an indication of [Ca2+]ᵢ signal. Data acquisition and preliminary analysis were done using FLIPR software (Molecular Devices). All calcium measurements were done at room temperature. The results are shown in FIG. 5.

The results showed that the recombinant rhP2X7 receptor from the stably transfected recombinant HEK293 cells showed the expected characteristics with respect to ligand kinetics, an EC₅₀ of about 3 mM for ATP and an EC₅₀ of about 150 μM for Bz-ATP.

### EXAMPLE 4

The expression of the rhP2X7 receptor in the stably transfected recombinant HEK293 cells was confirmed by TAQMAN real-time PCR analysis (Applied Biosystems). The following protocol was used. The forward PCR primer was h-P2X7-F1208 having the nucleotide sequence 5′-GCAAGTGCTG TCAGCC CTG-3′ (SEQ ID NO:8). The reverse PCR primer was h-P2X7-R1288 having the nucleotide sequence 5′-ATGTCGGCTT TGGCTCCA-3′ (SEQ ID NO:9). The PCR probe was rh-P2X7-F1229 having the nucleotide sequence 5′-TGGTCAACGA ATACTACTAC AGGAAGAAGT GCGAG-3′ (SEQ ID NO:10).

The Primers and fluorogenic probe were designed using the human P2X7 sequence (GenBank Accession No. NM_{—}002562) using Primer Express v. 1.0 (Applied Biosystems, Foster City, Calif.). The human and Rhesus monkey P2X7 nucleotide sequences are the same for the areas covered by the PCR primers and probe. The PCR Probe was synthesized by Applied Biosystems with the fluorescent reporter dye FAM (6-carboxy-fluorescein) attached to the 5′-end and the quencher dye TAMRA (6-carboxy-tetramethyl-rhodamine) attached to the 3′-end. Primers and probes were designed to span the intron-separating exons 10 and 11 as determined by comparing the cDNA sequence of the rhP2X7 with the genome DNA sequence of the human P2X7. The amplified PCR products were designed to be between 70 and 110 bp in length. For controls, human GAPDH PCR primers and probe were purchased from Applied Biosystems.

Reverse transcription reactions were carried out for each RNA sample prepared from stably transfected recombinant HEK293 cells in MICROAMP (Applied Biosystems) reaction tubes using TAQMAN reverse transcription reagents. Each reaction tube contained 250 ng of total RNA in a volume of 50 μL containing 1×TAQMAN RT buffer, 5.5 mM MgCl₂, 500 μM of each dNTP, 2.5 μM of oligo-d(T) 16 primers, random hexamers, 0.4 U/μL RNase inhibitor, and 1.25 U/μL MULTISCRIBE Reverse Transcriptase.

Reverse transcription reaction was carried out at 25° C. for 10 minutes, 48° C. for 30 minutes, and 95° C. for 5 minutes. The reverse transcription reaction mixture was then placed at 4° C. for immediate use for PCR amplification or stored at −20° C. for later use. Real-time TAQMAN PCR was performed in a MICROAMP Optical 96-well reaction plate (Applied Biosystems). For each 50 μL reaction, 10 μL of cDNA produced in the reverse transcription reaction (50 ng total RNA), 100 nM forward primer, 100 nM reverse primer, 200 nM probe, and 1× Universal Master Mix (Applied Biosystems) were combined. PCR amplification conditions were 2 minutes at 50° C. and 10 minutes at 95° C. followed by 40 cycles at 95° C. for 15 seconds and 60° C. for 1 minute. All reactions were performed in ABI Prism 7700 Sequence Detection System in duplicate using the SEQUENCE DETECTOR v 1.6 program (Applied Biosystems).

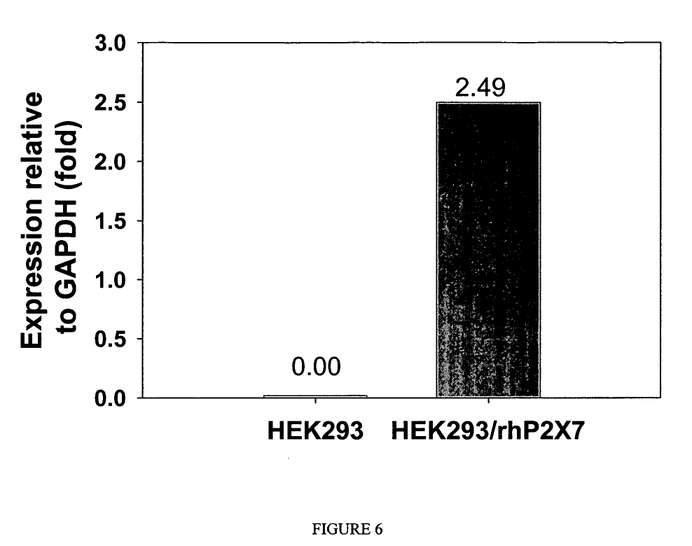

The results shown in FIG. 6 show that the recombinant HEK293 cells stably transfected with pcDNA5/FRT/TO/rhP2X7 produced mRNA encoding the rhP2X7 receptor.

Various clones of transfected cells were tested for expression of rhP2X7 protein. HEK293 wt cells; HEK293-rhP2X7 clone 300 cells; HEK293-rhP2X7 clone 212 cells; HEK293-rhP2X7 clone 424 cells; HEK293-rhP2X7 clone 430 cells; HEK293-rhP2X7 clone 630 cells; and HEK293-rhP2X7 clone 635 cells were separately cultured in phenol red free MEM medium supplemented with 10 mM Hepes, 2 mM L-glutamine, NEAA, 10% FBS, with or without 10 μM tetracycline, and penicillin/streptomycin and grown to 50-60% confluence. Cells were harvested after 24 hours incubation at 37° C. and frozen at −20° C. The frozen cell pellet was resuspended in 200 μL cell extraction buffer (50 mM Tris-HCL, pH8.0; 0.4 M NaCl, 5 mM EDTA, 1% Nonidet; 0.2% Sarcosyl; 100 μM Sodium Vanadate; 10 mM Sodium Molybdate; 20 mM NaF) containing a 1:100 dilution of protease inhibitor cocktail (Sigma) on ice, passed multiple times through a small micropipette tip, and incubated on ice for 30 minutes. The extract was centrifuged at 10,500×g for 30 minutes at 4° C. and supernatant fraction was transferred to a new tube. Protein concentration was measured using the BCA protein assay reagent A from Pierce (Rockford, Ill.). Cell extracts were subject to SDS PAGE (10%) and transferred to PVDF membranes. The membranes were blocked with blocking buffer (PBS, 10% glycerol, 0.2% TWEEN 20, and 10% non fat milk) for 1 hour. Blots were probed with antibodies against P2X7 overnight at 4° C. Blots were washed in blocking buffer and incubated with horseradish peroxidase-conjugated anti-rabbit or anti-mouse antibodies (Pierce, Rockford, Ill.). The membranes were washed and developed with SUPERSIGNAL West Dura ECL substrate (Pierce).

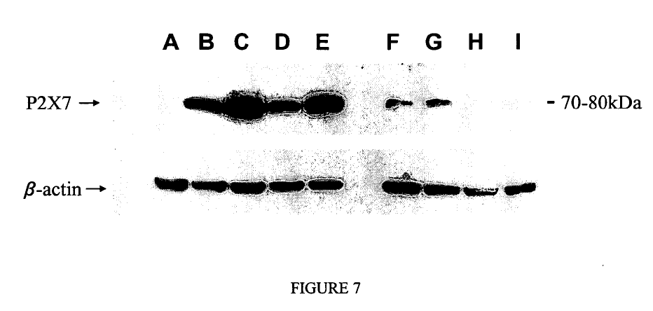

As shown in FIG. 7, HEK293-rhP2X7 clone 300 cells (lanes B and C) and HEK293-rhP2X7 clone 212 cells (lanes D and E); lane appeared to have the most robust expression of rhP2X7 protein.

### EXAMPLE 5

The cells that express the rhP2X7 receptor can be used in an assay to screen a library of analytes to identify those analytes that are P2X7 receptor modulators, e.g., agonists or antagonists. Therapeutic uses for analytes identified using the assay include use as disease modifying agents for osteoarthritis or in treatments for peripheral and neuropathic pain.

The following is an example of a calcium flux protocol that can be used in a high throughput screen using cells that express the rhP2X7 receptor. The cells are grown to about 75-85% confluence, rinsed with DPBS (5.33 mM KCl, 0.44 mM KH₂PO₄, 0.3 mM Na₂HPO₄, 4 mM NaHCO₃, 0.1% Glucose, 138 mM N-methyl glucamine), and dissociated with 0.05% trypsin. Then, about 20,000 cells are plated into multiple of the wells of multiwell plates in complete growth medium. The cells are incubated at 37° C. and 5% CO₂ for about 20 to 24 hours. Next, about 20 μL FLUO-4 loading buffer (Invitrogen) is added to each of the wells and the cells incubated at RT for 60 minutes. Then, about 10 μL aliquots of each analyte to be tested (final concentration in DMSO about 0.1%) diluted in TR-40 quenching solution (Sigma #T2397) is added to a well containing cells. Controls include antagonists such as KN-62 and Ox-ATP and agonists such as Bz-ATP (at an EC₈₀). The cells are incubated for about 10 minutes at RT. Next, about 10 μL of agonist (6× a,b-methylene ATP (EC₅₀=350 μM)) diluted in TR-40 quenching solution is added to each of the wells and changes in fluorescence is monitored using a FLEXSTATION (Molecular Devices) at room temperature.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

What is claimed:

1. A nucleic acid comprising a nucleotide sequence encoding a Rhesus monkey P2X7 (rhP2X7) receptor.

2. The nucleic acid of claim 1 wherein the rhP2X7 has the amino acid sequence of SEQ ID NO:2.

3. The nucleic acid of claim 1 wherein the nucleotide sequence comprises the nucleotide sequence of SEQ ID NO:1.

4. The nucleic acid of claim 1 wherein one or more of the nucleotide codons encoding the rhP2X7 receptor that occur at low frequency in nucleic acids encoding highly expressed proteins in humans have been replaced with nucleotide codons that occur at a higher frequency in the nucleic acids encoding the highly expressed proteins in humans.

5. An expression vector comprising the nucleic acid of claim 1 operably linked to a promoter.

6. A host cell containing the expression vector of claim 5 therein.

7. A process, comprising culturing the host cell of claim 6 in a cell culture medium under conditions for producing the rhP2X7 receptor.

8. A Rhesus monkey P2X7 (rhP2X7) receptor.

9. The rhP2X7 receptor of claim 8 comprising the amino acid sequence of SEQ ID NO:2.

10. A method of screening an analyte for its ability to modulate the activity of a mammalian P2X7 receptor comprising:
(a) contacting a recombinant, which expresses a Rhesus monkey P2X7 (rhP2X7) receptor, with a P2X7 receptor agonist, in the presence and absence of the analyte, and
(b) assaying for an alteration in the activity of the rhP2X7 receptor in the presence of the analyte, wherein a reduction or increase in the activity of the rhP2X7 receptor being indicative of an analyte that modulates mammalian P2X7 receptor activity.

11. The method of claim 10 wherein the agonist is ATP or Bz-ATP.

12. A method of screening a compound for its ability to enhance the activity of a mammalian P2X7 receptor comprising:
(a) contacting a recombinant cell, which expresses a Rhesus monkey P2X7 (rhP2X7) receptor, with the analyte;
(b) assaying for activity of the rhP2X7 receptor; and,
(c) comparing the activity with the activity of the rhP2X7 receptor present in the absence of the analyte, wherein an increase in the activity of the rhP2X7 receptor in the presence of the analyte is indicative of an analyte that enhances mammalian P2X7 receptor activity.

13. A method of screening an analyte for its ability to inhibit the activity of a mammalian P2X7 receptor comprising:
(a) contacting a recombinant cell, which expresses a Rhesus monkey P2X7 (rhP2X7) receptor, with the analyte and then with a P2X7 receptor agonist;
(b) assaying for activity of the rhP2X7 receptor; and,
(c) comparing the activity with the activity of the rhP2X7 receptor present in the absence of the analyte and in the presence of the agonist, wherein a decrease in the activity of the P2X7 receptor in the presence of the analyte is indicative of an analyte that inhibits P2X7 receptor activity.

14. The method of claim 13 wherein the agonist is ATP or Bz-ATP.

15. The method of claim 13 wherein the assaying is effected by monitoring the uptake into the recombinant cell of a detectable molecule.

16. The method of claim 14 wherein the detectable molecule is a fluorescent dye.

17. The method of claim 13 wherein the assaying is effected by measuring the intracellular concentration of Ca²⁺ in the cell.

18. The method of claim 15 wherein the recombinant cell is a HEK293 cell.

19. The method of claim 15 wherein the rhP2X7 receptor has the amino acid sequence of SEQ ID NO:2.


SEQUENCE LISTING (10 sequences)

SEQ ID NO: 1 - DNA, 1788 bp, Macaca mulatta
atgccggcctgctgcagctgcagtgatgttttccagtatgagacgaccaaagtcactcggatccagagcatgtattatggcaccattaagtggttcttccacgtgatcatcttttcctacgttagctttgctctggtgagtgacaagttgtaccagcggaaagagcctgtcatcagttctgtgcacactaaggtgaaggggatagcagaggtgaaagaggagatggtggagaatggagtgaagaagttggtgcacagtgtctttgacactgcagactacaccttccctttgcaggggaactctttcttcgtgatgacaaactttctcaaaacagaaggccaagagcagcggttatgtcctgagtatcccacccgcaggacgctctgttcctctgaccggggttgtaaaaagggatggatggacccgcagagcaaaggaattcagaccggaaggtgtgtagtgtatgaagggaaccggaagacctgtgaagtctctgcctggtgccccatcgaggcagtgggagaggccccccggcctgctctcttgaacagtgcggaaaacttcaccgtgctcatcaaaaacaatatcgacttccccggccacaactacaccacgagaaacatcttgcctggtttaaacatcacttgtaccttccacaagactcagaatccacagtgtcccattttccgactaggagacatcttccgagaaacaggcgataatttttcagatgtggcaattcagggcggaataatgggcattgagatctactgggactgcaacctagaccgttggttccatcactgccgtcccaaatacagtttccgtcgccttgatgacaagaccaccaacgtgtccttgtaccctggctacaacttcagatatgccaagtactacaaggaaaacaatgttgagcaacggactctgataaaagtctttgggatccgttttgacatcctggtttttggcaccggaggaaaatttgacattatccagctggttgtgtacatcggctcaaccctctcctacttcggtctggccactgtgttcattgacttcctcatcaacacttactccagtaactactgtcgctcccatatttatccctggtgcaagtgctgtcagccctgtgtggtcaacgaatactactacaggaagaagtgcgagtccattgtggagccaaagccgacattaaagtatgtgccctttgtggatgaatcccacattaggatggtgaaccagaagctactagggagaagtctgcaagatgtcaagggccaagaagtcccaagacctgcaatggacttcacagatttgtccaagctgcccctggccctccatgacccacccccgattcctggacaaccaggggagatgcagccgcttagagaggaggcgactcctagatccagggacagccccgtctggtgccagtgtggaagctgccttccgtcccaactccccaagagccacaggtgcctggaggagctgtgctgccggaaaaagccaggggcctgcatcaccacctcagagctgttcaggaagctggtcctgtccagacacgtcctgcagttcctcctgctctaccaggagcccttgctggcgctggatgtggattccaccaacagccggctgcgacactgtgcctacaggtgctatgccacctggcgcttcggctcccaggacatggctgaccttgccatcctgcccagctgctgccgctggaggatccgaaaagagtttcccaagagtgaagggcagtacagtggcttcaagagtccttactga

SEQ ID NO: 2 - protein, 595 aa, Macaca mulatta
MPACCSCSDVFQYETTKVTRIQSMYYGTIKWFFHVIIFSYVSFALVSDKLYQRKEPVISSVHTKVKGIAEVKEEMVENGVKKLVHSVFDTADYTFPLQGNSFFVMTNFLKTEGQEQRLCPEYPTRRTLCSSDRGCKKGWMDPQSKGIQTGRCVVYEGNRKTCEVSAWCPIEAVGEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEQRTLIKVFGIRFDILVFGTGGKFDIIQLVVYIGSTLSYFGLATVFIDFLINTYSSNYCRSHIYPWCKCCQPCVVNEYYYRKKCESIVEPKPTLKYVPFVDESHIRMVNQKLLGRSLQDVKGQEVPRPAMDFTDLSKLPLALHDPPPIPGQPGEMQPLREEATPRSRDSPVWCQCGSCLPSQLPKSHRCLEELCCRKKPGACITTSELFRKLVLSRHVLQFLLLYQEPLLALDVDSTNSRLRHCAYRCYATWRFGSQDMADLAILPSCCRWRIRKEFPKSEGQYSGFKSPY

SEQ ID NO: 3 - DNA, 34 bp, Artificial Sequence
  PCR primer P2X7uphind
cgcaagcttatgccggcctgctgcagctgcagtg

SEQ ID NO: 4 - DNA, 33 bp, Artificial Sequence
  PCR primer P2X7dohind
cgcaagctttcagtaaggactcttgaagccact

SEQ ID NO: 5 - DNA, 33 bp, Artificial Sequence
  PCR primer P2X7ecomidup
cgcgaattcctttgctctgcgggtccatccatc

SEQ ID NO: 6 - DNA, 36 bp, Artificial Sequence
  PCR primer P2X7ecomiddo
cgcgaattcagaccggaaggtgtgtagtgtatgaag

SEQ ID NO: 7 - DNA, 6931 bp, Artificial Sequence
  pcDNA5/FRT/TO/rhP2X7
  misc_feature (232)...(958): CMV promoter
  misc_feature (984)...(2771): rhP2X7
  misc_feature (2889)...(3113): BGH poly A signal
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttatgccggcctgctgcagctgcagtgatgttttccagtatgagacgaccaaagtcactcggatccagagcatgtattatggcaccattaagtggttcttccacgtgatcatcttttcctacgttagctttgctctggtgagtgacaagttgtaccagcggaaagagcctgtcatcagttctgtgcacactaaggtgaaggggatagcagaggtgaaagaggagatggtggagaatggagtgaagaagttggtgcacagtgtctttgacactgcagactacaccttccctttgcaggggaactctttcttcgtgatgacaaactttctcaaaacagaaggccaagagcagcggttatgtcctgagtatcccacccgcaggacgctctgttcctctgaccggggttgtaaaaagggatggatggacccgcagagcaaaggaattcagaccggaaggtgtgtagtgtatgaagggaaccggaagacctgtgaagtctctgcctggtgccccatcgaggcagtgggagaggccccccggcctgctctcttgaacagtgcggaaaacttcaccgtgctcatcaaaaacaatatcgacttccccggccacaactacaccacgagaaacatcttgcctggtttaaacatcacttgtaccttccacaagactcagaatccacagtgtcccattttccgactaggagacatcttccgagaaacaggcgataatttttcagatgtggcaattcagggcggaataatgggcattgagatctactgggactgcaacctagaccgttggttccatcactgccgtcccaaatacagtttccgtcgccttgatgacaagaccaccaacgtgtccttgtaccctggctacaacttcagatatgccaagtactacaaggaaaacaatgttgagcaacggactctgataaaagtctttgggatccgttttgacatcctggtttttggcaccggaggaaaatttgacattatccagctggttgtgtacatcggctcaaccctctcctacttcggtctggccactgtgttcattgacttcctcatcaacacttactccagtaactactgtcgctcccatatttatccctggtgcaagtgctgtcagccctgtgtggtcaacgaatactactacaggaagaagtgcgagtccattgtggagccaaagccgacattaaagtatgtgccctttgtggatgaatcccacattaggatggtgaaccagaagctactagggagaagtctgcaagatgtcaagggccaagaagtcccaagacctgcaatggacttcacagatttgtccaagctgcccctggccctccatgacccacccccgattcctggacaaccaggggagatgcagccgcttagagaggaggcgactcctagatccagggacagccccgtctggtgccagtgtggaagctgccttccgtcccaactccccaagagccacaggtgcctggaggagctgtgctgccggaaaaagccaggggcctgcatcaccacctcagagctgttcaggaagctggtcctgtccagacacgtcctgcagttcctcctgctctaccaggagcccttgctggcgctggatgtggattccaccaacagccggctgcgacactgtgcctacaggtgctatgccacctggcgcttcggctcccaggacatggctgaccttgccatcctgcccagctgctgccgctggaggatccgaaaagagtttcccaagagtgaagggcagtacagtggcttcaagagtccttactgaaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 8 - DNA, 19 bp, Artificial Sequence
  Forward PCR Primer h-P2X7-F1208
gcaagtgctgtcagccctg

SEQ ID NO: 9 - DNA, 18 bp, Artificial Sequence
  Reverse PCR primer h-P2X7-R1288
atgtcggctttggctcca

SEQ ID NO: 10 - DNA, 35 bp, Artificial Sequence
  PCR probe rh-P2X7-F12295
tggtcaacgaatactactacaggaagaagtgcgag